(19) United States
(12) Patent Application Publication
(10) Pub. No.: US 2009/0214466 A1
(43) Pub. Date: Aug. 27, 2009

(54) Methods of Alleviating Disorders and Their Associated Pain

(76) Inventor: Bruce H. Levin, Philadelphia, PA (US)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 11/913,914

(22) PCT Filed: May 9, 2006
(86) PCT No.: PCT/US06/17924

§ 371 Date: Oct. 13, 2008

(51) Int. Cl. A61K 38/20; A61K 39/395; A61K 39/08; A61K 38/16; A61K 31/505; A61K 31/404; A61K 31/40; A61K 31/366; A61K 31/216; A61K 31/192; A61K 31/132

(52) U.S. Cl. 424/85.2; 424/138.1; 424/247.1; 514/12; 514/275; 514/419; 514/423; 514/460; 514/532; 514/571; 514/674

(57) ABSTRACT

The invention relates generally to the field of treatment of headache, post-laminectomy syndrome, and other disorders associated with localized pain. The compositions and methods described herein are useful for alleviating both the disorders and the pain associated therewith.

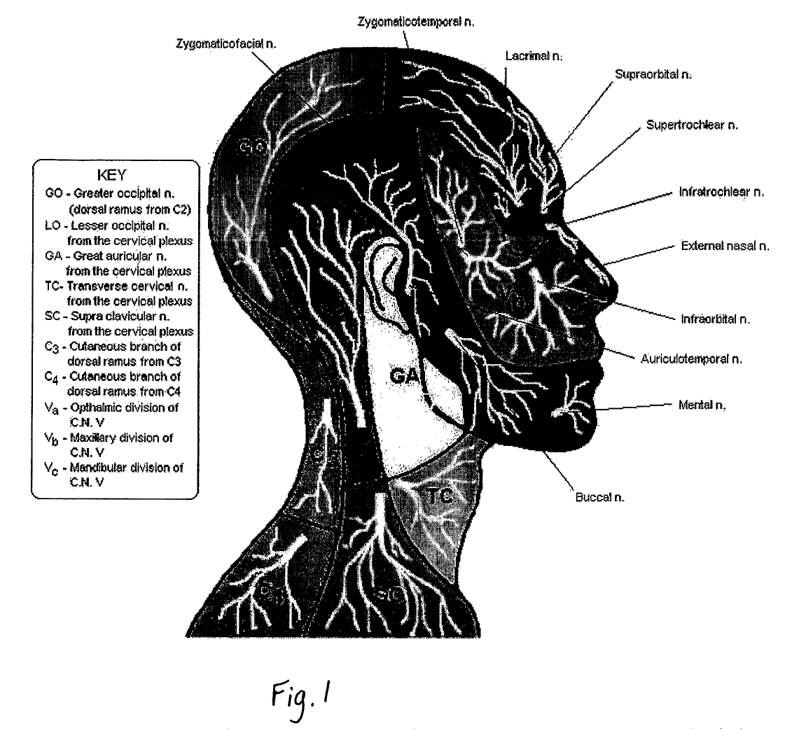

### RELATED APPLICATIONS

This application is a national phase application of PCT/US2006/017924, filed 9 May 2006, and is entitled to priority pursuant to 35 USC 119(e) to U.S. provisional application No. 60/679,028, filed 9 May 2005.

### BACKGROUND OF THE INVENTION

The invention relates generally to the field of treatment of headache.

There are currently a very large number of headache syndromes recognized by the International Headache society and other professional organizations. In general headaches are classified as to their symptomatology, associated symptoms, frequency, pattern and other features. The treatment of headaches are often determined by current classification. The inventor discloses a more effective approach to classification and offers approaches which yield more effective therapeutic results

### DETAILED DESCRIPTION OF THE INVENTION

It has long been suggested that vasodilation is associated with headache pain. Recent evidence suggests that ischemia may indeed be important in headache pathophysiology. Also recognized are the roles of neurogenic inflammation and central and peripheral neural sensitization.

The inventor proposes that headaches be divided into classes according to their primary generators. For example, direct central, associated central and autonomic, toxic/metabolic, hormonal and peripheral somatic, and peripheral neural primary limbs may be the primary headache generators for a given headache class. It is well known that peripheral neural input modulates and effects changes the actual anatomic structures and physiologic function of the in many states. Pain or loss of limb changes the actual anatomy of the subserved portions of the brain. It is also know that central neurologic changes affect the peripheral neural structures and their function. Without being bound by any specific theories, the inventor holds that it is a complex interaction between central, peripheral, and other factors which modulates headache symptoms and pathophysiology. Once the generator threshold has been lowered in a peripheral or central generator, any subsequent assault on that generator by any of the above referenced factors will trigger a headache.

For example, many migraines are generated from the cervical spine and related structures, often the occipital nerves. Once the structures are irritated or otherwise stimulated, the central nervous system may react with classic migraine physiology. In another person, this may present as a classic cluster or other type of headache. Hence, two or more discreet types of headaches may have the same generator. Of course, hormonal fluctuations which accompany menstrual cycle changes may further lower the stimulation threshold, making the headache cyclical or in other ways related to hormonal changes. Further, the inventor holds that once usual headache generators are stimulated, other normally quiescent structures may become stimulated and in turn irritate the primary generator.

For example, the inventor views the CNS as exhibiting forward and backward feedback to and from the occipital nerves, supraorbital nerves, infraorbital nerves, supratrochlear nerves, auriculotemporal nerves, sphenoplatine ganglion, and other neural structures. Therefore, stimulation of the SPG may trigger a migraine which may produce classic symptoms, or the supraorbital nerve may do the same.

The inventor has witnessed a patient who had one type of headache disappear with ipsilateral third occipital nerve block, only to recur on the opposite side which it had not done before. When this headache was subsequently treated with a third occipital block on the other side, the patient lost that headache and developed a headache in the distribution of the supraorbital nerve. This headache went away with supraorbital nerve block.

Hence, once the CNS has been excited and generator threshold lowered, any peripheral generator may trigger a new type of headache as documented here. As such, the inventor proposes headache classification based on the location of the pain. Pain in, behind, or around the eye is related to dysfunction of the supraorbital, supratrochlear, or occipital nerves. Headaches in the lower orbit may be triggered by the infraorbital nerve. Ocular, frontal, or even some occipital headaches may be mediated by the SPG, while occipital headaches are mediated by the occipital nerves, facet joints (i.e., zygapophysial joints), third occipital nerve, atlantoaxial/atlantooccipital nerves and so on. Hence, attempts should be made to classify headaches initially classified as to their origin and location. Of course metabolic headaches, infectious headaches, and sinus headaches will be treated separately as will headaches from brain scarring. Coronal headaches may also not be easily classified according to this scheme.

According to this scheme, novel treatment options are proposed to maximize effectiveness and minimize systemic side effects.

Treatment options include the following.

1) Application of any local anesthetic agent or compound to any of the areas described in Table 1 by a method in compliance with the aforementioned diagnostic/classification scheme, including:

a) direct injection in series or parallel with or without any of the agents listed below

b) iontopheresis, patch (e.g., Lidoderm® or a similar patch), cream, liquid or emulsion (e.g., EMLA), alone or in combination with any of the agents listed below

TABLE I
A List of Selected Head and Neck Nerves

| | |
|---|---|
| Some Recognized | Olfactory, Optic, Oculomotor, Trochlear, Trigeminal, Abducent, |
| Nerves of the Head | Facial, Vestibulocochlear, Glossopharyngeal, Vagus, Cranial |
| | accessory, Spinal accessory, Hypoglossal, Zygomaticofacial, |
| | Zygomaticotemporal, Lacrimal, Supraorbital, Supertrochlear, |
| | Intratrochlear, External nasal, Infraorbital, Auriculotemporal, |
| | Mental, Buccal, and opthalmic, maxillary and, mandibular |
| | divisions of C.N.V |
| Some Recognized | The cervical nerves, including the cervical plexus and the anterior |
| Nerves of the Neck | roots of C1-C4, Phrenic, Brachial Plexus (C5-T1), posterior rami of |
| | cervical nerves, Greater occipital, Lesser Occipital, Great auricular, |
| | Transverse cervical, Supraclavicular, Cutaneous branches of dorsal |
| | rami from C3 and C4 |

2) Administration of one or more drugs or agents which decrease serum lipids or cholesterol or alter the serum ratio of HDL/LDL to effect a decrease in inflammatory activity. This method is effective for decreasing the symptoms and pathophysiology of one or more of headaches, migraines, cluster headaches, trigeminal neuralgia, central pain, neuropathic pain, peripheral neuropathy, radiculopathies, diabetic neuropathy, chronic regional pain syndrome, toxic neuropathies, metabolic neuropathies, failed back/neck syndrome, back pain, arthritis, immunologic disorders (including scleroderma, rheumatoid arthritis, and lupus, for example) Crohn's disease, ulcerative colitis, multiple sclerosis, Huntington's disease, Alzheimer's disease, Lyme disease, poor sleep, jet lag, anxiety /depression, and ADD. Such drugs can include:

A) Statins such as:

Atorvastatin (LIPITOR®)

Fluvastatin (LESCOL®)

Lovastatin (MEVACOR®)

Pravastatin (PRAVACHOL®)

Rosuvastatin Calcium (CRESTOR®)

Simvastatin (ZOCOR®);

B) Binding compounds or resins which decrease total fats or cholesterol such as:

Cholestyramine (QUESTRAN®, PREVALITE®, LO-CHOLEST®)

Colestipol (COLESTID®)

Colesevelam (WELCHOL®);

C) Drugs affecting HDL/LDL and triglyceride ratios such as:

Clofibrate (ATROMID-S®) & Gemfibrozil (LOPID®);

D) Niacin, or other B vitamins, including folate;

E) Neutraceuticals including oats, bran, chitin and other agents known in the industry to lower fats, lipids and cholesterol;

F) Anti-inflammatory fatty acids such as safflower oil, fish oil, omega oils, and cetyl myristoleate;

3) Administration of botulinum toxin (including types A, B, C, D, E, F, G, and H) has been described as useful in a variety of disorders, but the route of administration is in muscle, into the neuromuscular junction, or into joint synovial fluid. I Claim that injection of these or similar compounds directly into peripheral, central, or other nerves, including the third occipital nerve, sphenopalatine ganglion, trigeminal nerve, spinal accessory nerves, medial branch nerve(s), or similar neural structures, the cervicothoracic, thoracic, lumbosacral autonomic nerve ganglia, the ganglia impar, coeliac plexus, superior/inferior hypogastric nerves/plexuses, or into deep brain structures including the periaqueductal grey, scar areas, CNS pain generators, or intraventricular, epidural, or subdural placement will increase efficacy and decrease side effects. A depot composition utilizing common compounds currently used for depot medications, or micelles, liposomes, protein compounds, nanotech carbon, or other long term or extended release mechanisms is also claimed. Release can also be modulated by an applied energy field such as radio waves, radiofrequency, light, heat, magnetic, microwave, sound, electric, or other form of energy applied to the depot compound, or internal reservoir, and such modulated release is also within the scope of what is claimed.

The diseases which can be treated in the manner described herein include headache, migraine, cluster headache, trigeminal neuralgia, postherpetic neuralgia, failed back/neck syndrome, chronic regional pain syndrome, thalamic pain syndromes, central pain syndromes, peripheral neuropathies, post spinal cord injury pain, phantom pain, sympathetic mediated pain, diabetic neuropathies, chronic abdominal, pelvic, genitourinary, or rectal pain, as well as seizures and manic depressive, anxiety, and schizophrenic or other psychotic disorders.

4) Treatment of migraine, cluster headaches, chronic daily headaches can be achieved as described herein with the following anti-inflammatory agents:

TNF antagonists including but not limited to: etanercept (ENBREL®, Immunex Corporation); infliximab (REMICADE®, Johnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and a molecule containing at least one soluble TNF receptor.

Such agents can also include antagonists of one or more of the following: interleukin-I (IL-1), IL-6, TNF-alpha, TGF-Beta; agonists of one or more of the following: IL-4, IL-10, and IL-13 agonists; and antagonists of one or more of the following: LIF, IFN-gamma, OSM, CNTF, TGF-beta, GM-CSF, IL-11, IL-12, IL-17, IL-18, IL-8 tachykinins, VIP (vasoactive intestinal peptide), and VPF (vascular permeability factor), caspase-1, caspase-5, PYCARD, NALP1, the SIS family of cytokines, the SIG family of cytokines, the SCY family of cytokines, the platelet factor-4 superfamily of intercrines, and prostaglandins. All Dosing units are as per standard dosing regimens.

These agents can be injected into the nerve structures listed in Table 1 for the treatment of migraine, cluster headache or tension headache.

These agents can be injected epidurally or intrathecally or to the facet or related joints or into the sphenopalatine ganglion or targeted intranasal structures.

5) Treatment of any inflammatory disorder with a Cox-2 agent such as VIOXX®, CELEBREX®, BEXTRA®, PREXIGE® or ARCOXIA® in combination with an anti-platelet or anti-coagulant agent such as aspirin, PLAVIX®, TICLID®, RHEOPRO®, AGGRASTAT®, AGGRENOX®, PERSANTINE®, INTEGRILIN®, coumadin, anti-clotting factor, NSAIDS, extracts of garlic, ginger, cumin, onions, turmeric, or Chinese black tree fungus, and vitamins C and E to decrease risk of cardiovascular side effects

6) Treatment of migraine, cluster headaches, chronic daily headaches with NSAID, Cox-2 or -3 agents noted above by perineuronal, peripheral neural, facet, or epidural injection-alone or in combination with another agent disclosed in this application.

7) Treatment of migraine and cluster headaches with systemic steroids, intrathecal steroids, epidural steroids, intra- or peri-facet steroids, or occipital steroids or steroids applied to structures referenced in Table 1.

8) Treatment of failed back/neck syndrome with anti-inflammatory compounds other than steroids, any C. botulinum compounds (i.e., C. botulinum toxin or active fragments thereof), NSAID compounds—alone or in combination with each other or with a steroid compound.

The practice of the method above can be performed using ultrasound, X-ray, fluoroscope-guided, CT-Guided, or MRI-guided imaging to maximize precise targeted non-intravascular and non-intraneural placement while avoiding nerve trauma with said agents. Contrast dye compounds may be administered concurrently as a new formulation or given prior to injection of medication to verify optimal placement. This guidance can enhance and/or verify desired placement of medication. Such guidance can be used, for example, to achieve:

A) Transforaminal placement

B) Intra-facet placement

C) Medial branch nerve targeted placement.

D) Sympathetic ganglion placement

9) Treatment of any syndrome associated with peri-neural scar tissue formation using any of the compounds or methods described herein.

10) Treatment of degenerative disc disease, spondylosis and spinal stenosis using any of the compounds or methods described herein.

11) Utilizing injectable or locally administered, patch administered anti-seizure agents such as neurontin, anti-spasmodics (e.g., ZANAFLEX®, anti-depressant, serotonin uptake inhibitor, NMDA antagonist (e.g., ketamine or dextromethorphan)—alone or in combination—for the treatment of migraine, cluster headache, chronic daily headache, peripheral neuropathy, neuropathic pain syndrome, radiculopathy, chronic regional pain syndrome, arthritis, failed back/neck syndrome, chronic abdominal, pelvic, genitourinary or rectal pain syndromes, or for cancer pain syndromes.

12) The use of an implantable pump system to administer drugs or drug classes referenced in one or more of items 3), 4), 5), and 11) to the epidural, intrathecal, perineural, or periventricular spaces, or into autonomic ganglia.

13) The use of polyethylene glycol for the treatment of one or more of neuropathy, radiculopathy, headache migraine, peripheral neuropathy, traumatic neuropathy, chronic regional pain syndrome, failed back/neck syndrome, and arthritis. For example, PEG can be administered by injection perineurally, intra-articularly, periarticularly, intrathecally, epidurally, into or around sympathetic ganglia, or transforaminally.

14) Erythropoietin can also be administered as described in item 13) herein.

15) The invention also includes treatment by injection into intraarticular, periarticular, perineural, intrathecal, epidural, intraocular, periventricular, or into or near other structures of an anti-inflammatory agent such as one or more TNF antagonists such as etanercept (ENBREL®, Immunex Corporation); infliximab (REMICADE®, Johnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and a molecule containing at least one soluble TNF receptor; one or more antagonists of a compound such as IL-1, IL-6, TNF-alpha, TGF-Beta; agonists of one or more of the following: IL-4, IL-10, and IL-13 agonists; and antagonists of one or more of the following: LIF, IFN-gamma, OSM, CNTF, TGF-beta, GM-CSF, IL-11, IL-12, IL-17, IL-18, IL-8 tachykinins, VIP (vasoactive intestinal peptide), and VPF (vascular permeability factor), caspase-1, caspase-5, PYCARD, NALP1, the SIS family of cytokines, the SIG family of cytokines, the SCY family of cytokines, the platelet factor-4 superfamily of intercrines, and anti-prostaglandins—alone or in combination with one or more bactericides or bacteriostats such as silver or metal ions, antifungal agents, and antibiotic agent such as a tetracycline, penicillin with or without clavulanic acid compound, cephalosporin, gentamicin, tobramycin, vancomycin, ciprofloxacin, and other effective antimicrobial agent known to one skilled in the art to prevent local or systemic infections which may accompany the use of anti-inflammatory agents.

The agents described herein can be co-administered with a local anesthetic to further decrease neurogenic inflammation and pain.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

A middle-aged woman complained of migraine symptoms. A standard dose of ENBREL® etanercept 50 milligrams was administered to the patient as described herein. Reduction in the frequency and severity of migraine symptoms was reported.

### Example 2

A middle-aged woman complained of migraine symptoms. A standard dose of BOTOX® botulinum toxin type A (ca. 100 units) was administered as described herein to the occipital nerve. Reduction in the frequency, severity, and duration of migraine symptoms was reported.

### Example 3

A middle-aged man complained of symptoms of post-laminectomy syndrome. A standard dose of REMICADE® infliximab was administered by intramuscular injection. The patient reported reduced symptoms.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method of alleviating localized pain associated with a disorder in a patient, the method comprising administering to the patient an anti-inflammatory agent in an amount effective to suppress inflammation at a first site on a nerve that enervates the body location at which the pain occurs, wherein the first site is located along the nerve between the spinal cord and the body location.

2. The method of claim 1, wherein the anti-inflammatory agent is locally administered at the first site.

3. The method of claim 1, further comprising locally administering a local anesthetic to the nerve at a second site located along the nerve between the spinal cord and the body location in an amount effective to anesthetize the nerve.

4. The method of claim 3, wherein the first site and the second site are the same site.

5. The method of claim 3, wherein the anti-inflammatory agent is administered systemically.

6. The method of claim 1, wherein the anti-inflammatory agent is topically administered to a skin surface enervated by the nerve.

7. The method of claim 1, wherein the anti-inflammatory agent is locally administered to a body location distinct from the body location at which the pain occurs.

8. The method of claim 7, wherein the disorder is a migraine.

9. The method of claim 8, wherein the anti-inflammatory agent is locally administered to a nerve at a cervical facet joint.

10. The method of claim 8, wherein the anti-inflammatory agent is injected into a cervical facet joint.

11. The method of claim 7, wherein the disorder is a post-laminectomy syndrome.

12. The method of claim 11, wherein the anti-inflammatory agent is locally administered to a nerve at a spinal facet joint.

13. The method of claim 11, wherein the anti-inflammatory agent is injected into a spinal facet joint.

14. The method of claim 11, wherein the anti-inflammatory agent is injected into a spinal neuroforamen.

15. The method of claim 1, wherein the anti-inflammatory agent is a tumor necrosis factor (TNF) antagonist.

16. The method of claim 15, wherein the TNF antagonist is selected from the group consisting of anti-TNF antibodies, soluble TNF receptors, and combinations of these.

17. The method of claim 1, wherein the anti-inflammatory agent is not a steroid compound.

18. The method of claim 1, wherein the anti-inflammatory agent is a compound which modulates serum lipid levels.

19. The method of claim 18, wherein the agent is selected from the group consisting of statins, cholesterol-binding materials, and combinations of these.

20. The method of claim 18, wherein the agent is selected from the group consisting of atorvastatin, fluvastatin, lovastatin, pravastatin, rosuvastatin, simvastatin, and combinations of these.

21. The method of claim 18, wherein the agent is selected from the group consisting of cholestyramine, colestipol, colesevelam, and combinations of these.

22. The method of claim 18, wherein the agent is selected from the group consisting of clofibrate, gemfibrozil, and combinations of these.

23. The method of claim 1, wherein the anti-inflammatory agent is selected from the group consisting of safflower oil, fish oil, cetyl myristoleate, and combinations of these.

24. The method of claim 1, wherein the anti-inflammatory agent is an cytokine.

25. The method of claim 1, wherein the anti-inflammatory agent is selected from the group consisting of cytokine antagonists, cytokine agonists, and combinations of these.

26. The method of claim 1, wherein the anti-inflammatory agent is an antagonist of a cytokine selected from the group consisting of interleukin-1 (IL-1), IL-6, TNF-alpha, and tumor growth factor beta.

27. The method of claim 1, wherein the anti-inflammatory agent is an agonist of a cytokine selected from the group consisting of IL-4, IL-10, and IL-13.

28. The method of claim 1, further comprising administering an anti-coagulant to the patient in an amount sufficient to inhibit blood clotting.

29. The method of claim 285, wherein the anti-inflammatory agent comprises a cox-2 inhibitor.

30. A method of alleviating localized pain associated with a disorder in a patient, the method comprising locally administering an active portion of a botulinum toxin to the patient at a first site on a nerve that enervates the body location at which the pain occurs in an amount effective to alleviate pain, wherein the first site is located along the nerve between the spinal cord and the body location.

31. The method of claim 30, wherein the active portion is the entire botulinum toxin.

32. The method of claim 30, wherein the botulinum toxin is selected from the group consisting of botulinum toxins A, B, C, D, E, F, G, H, and combinations of these.

33. The method of claim 30, wherein the disorder is selected from the group consisting of a migraine, a cluster headache, and a post-laminectomy syndrome.

34. A method of alleviating a disorder associated with in localized pain a patient, the method comprising locally administering an active portion of a botulinum toxin to the patient at a first site on a nerve that enervates the body location at which the pain occurs in an amount effective to alleviate the disorder, wherein the first site is located along the nerve between the spinal cord and the body location.

35. A method of alleviating a disorder associated with localized pain in a patient, the method comprising administering to the patient an anti-inflammatory agent in an amount effective to suppress inflammation at a first site on a nerve that enervates the body location at which the pain occurs, wherein the first site is located along the nerve between the spinal cord and the body location.

36. A method of alleviating localized pain associated with a disorder in a patient, the method comprising administering to the patient an anti-inflammatory agent in an amount effective to alleviate the pain.

37. A method of alleviating a disorder associated with localized pain in a patient, the method comprising administering to the patient an anti-inflammatory agent in an amount effective to alleviate the disorder.

38. The method of claim 37, wherein the disorder is selected from the group consisting of a migraine, a cluster headache, and a post-laminectomy syndrome.

39. A method of alleviating localized pain associated with a disorder in a patient, the method comprising locally administering to the patient an anti-inflammatory agent in an amount effective to alleviate the pain.

40. A method of alleviating a disorder associated with localized pain in a patient, the method comprising administering to the patient an anti-inflammatory agent in an amount effective to alleviate the disorder.

41. The method of claim 40, wherein the disorder is selected from the group consisting of a migraine, a cluster headache, and a post-laminectomy syndrome.
